(19) United States
(12) Patent Application Publication
(10) Pub. No.: US 2016/0009856 A1
(43) Pub. Date: Jan. 14, 2016

(54) MANUFACTURING METHOD OF LACTIDE FROM LACTIC ACID

(71) Applicant: HYUNDAI MOTOR COMPANY, Seoul (KR)

(72) Inventors: Chae Hwan Hong, Ansan (KR); Si Hwan Kim, Anyang (KR); Ji Youn Seo, Suwon (KR); Do Suck Han, Seongnam (KR)

(21) Appl. No.: 14/859,718

(22) Filed: Sep. 21, 2015

Related U.S. Application Data
(63) Continuation-in-part of application No. 13/181,808, filed on Jul. 13, 2011.

(30) Foreign Application Priority Data
May 26, 2011 (KR) 10-2011-0050307

(51) Int. Cl. C08G 63/08; C07D 319/12

(52) U.S. Cl. CPC C08G 63/08; C07D 319/12

(57) ABSTRACT

The present invention provides a method of producing D-type lactide from liquid D-type lactic acid, and a method for producing D-type polylactic acid having a weight average molecular weight of about 50,000˜20,000 g/mol from the produced D-type lactide. The method of the present invention is advantageous in that D-type lactide can be obtained at a high yield by a simple method, compared to the conventional production methods. Consequently, production cost of D-type polylactic acid that is finally obtained from D-type lactide can be reduced.

### CROSS-REFERENCE TO RELATED APPLICATION

The present application is a continuation-in-part application of U.S. patent application Ser. No. 13/181,808 filed Jul. 13, 2011, which claims under 35 U.S.C. §119(a) the benefit of Korean Patent Application No. 10-2011-0050307 filed May 26, 2011, the entire contents of which are incorporated by reference herein.

### BACKGROUND

(a) Technical Field

The present invention relates to a method of producing a high yield of lactide from D-type lactic acid monomers produced by fermentation.

(b) Background Art

From the 20th century till now, the rapid industrialization has contributed to the consumption of fossil fuels, in particular, petroleum, and the growing demand for petroleum has been driven by rapid industrialization and population growth. However, petroleum is not renewable but rather a natural resource with a limited reserve which is soon to be depleted. In addition, carbon dioxide emissions generated by the burning of fossil fuels appears to be the main cause of global warming In this regard, there has been growing attention on improvement of energy-efficiency and alternatives to replace petroleum in order to reduce carbon dioxide emissions.

A plant-derived, biomass polymer is prepared from renewable plant resources such as corn, soybean, sugarcane, and wood by a chemical or biological method, and it is important in terms of less environmental impact by reducing carbon dioxide emissions rather than biodegradability. Among the biomass polymers, polylactic acid is a linear aliphatic polyester, and prepared by starch fermentation of corn and potato or by polymerization of sugar monomers that are obtained from glycosylation and fermentation of plant cellulose. It is also a carbon-neutral, environment-friendly, thermoplastic polymer resource.

Although polylactic acid has many advantages, it has a low impact resistance and low heat distortion temperature, compared to petrochemical polymers, thus impeding its to direct application into the automotive industry. In particular, polylactic acid has a low impact strength due to its brittle nature, and thus its application into materials for automotive parts is hindered. Due to the rather inferior physical properties to those of general polymer materials, polylactic acid resin has a very limited industrial application. In fact, improvement of physical properties is essential for automotive engine and chassis applications which require high heat resistance and impact resistance. To solve these problems, there has been introduced a technique to produce a stereo-complex resin by blending optical isomer resins.

In order to develop the technique to produce a stereo-complex resin, it is important to secure a method of producing L-type and D-type polylactic acids. Currently, commercial production of L-type polylactic acid has been actively carried out, but production of D-type polylactic acid is still at an early stage of development. Therefore, there is an urgent need to develop a cost-effective technique for the production of D-type polylactic acid resin.

Lactic acid has one asymmetric carbon atom and thus can be found in two enantiomeric forms. Lactide, on the other hand, has two asymmetric carbon atoms and can be found in three steroisomeric forms: L-lactide in which both asymmetric carbon atoms possess the L (or S) configuration; D-lactide in which both asymmetric carbon atoms possess the D (or R) configuration; and meso-lactide in which one asymmetric carbon atom has the L configuration and the other has the D configuration.

L-lactide and D-lactide are enantiomers. In the production of lactide from lactic acid, it would be advantageous if the absolute configuration of the lactic acid feed was maintained in its conversion to lactide. Therefore, production of lactide has proceeded by the initial formation of oligomeric lactic acid, LnA, such as by dehydration of aqueous lactic acid, followed by a catalytic transesterification reaction known as back-biting

Catalysts proposed for this reaction include tin powder, tin halides, or tin carboxylates (EP Publication 261,572 and 275,581); tin alkoxides (U.K. Pat. No. 1,007,347); and zinc or tin (EP Publication 264,926, and U.S. Pat. No. 4,797,468).

Additional processes include U.S. Pat. No. 4,797,163 which discloses a process of producing lactide by heating an alkali or alkaline earth metal salt of 2-halopropionic acid in a non-aqueous solvent, and U.S. Pat. No. 4,070,375 which discloses a process of preparing 1,4-dioxan-2-one and 5-substituted-1,4-dioxan-2-one by contacting CO with formaldehyde, 1,2-glycol, and HF catalyst.

U.S. Pat. No. 4,727,163 describes that a thermally stable polyether core/a-hydroxy acid (ester) block copolymer is thermally degraded under vacuum to form cyclic esters. U.S. Pat. No. 4,835,293 describes a process for preparing cyclic esters by cracking a prepolymer comprising an α-hydroxy acid (ester) polymer or a block copolymer thereof on a stable polyether in the presence of an inert gas at atmospheric or superatmospheric pressure, in which the cyclic ester is carried from the reaction with the inert gas to a solvent system.

However, even with the above advances, it is still not clear whether these methods produce high yields and provide cost-effective processes.

The above information disclosed in this Background section is only for enhancement of understanding of the background of the invention and therefore it may contain information that does not form the prior art that is already known in this country to a person of ordinary skill in the art.

### SUMMARY OF THE DISCLOSURE

The present invention provides a process for producing cyclic lactide by back-biting of low molecular weight polylactic acid chain, in which low molecular weight polylactic acid is formed by polymerization of liquid phase lactic acid, followed by depolymerization. In particular, the present invention provides a technique of producing lactide from a liquid phase lactic acid and a complex conversion process of lactide through alumina catalyst reaction of linear lactic acid dimer and trimer in gas phase, in which degree of polymerization and depolymerization properties are precisely controlled when low molecular weight polymerization of liquid phase lactic acid and depolymerization are performed.

In an exemplary embodiment of the present invention a method of producing D-type lactide begins by converting liquid D-type lactic acid into D-type polylactic acid having a weight average molecular weight of about 600˜1200 g/mol at a temperature of about 130˜150° C. and a pressure of 10˜200 torr. Next, the D-type polylactic acid having a weight average molecular weight of about 600˜1200 g/mol is converted into a gas stream by heating the D-type polylactic acid in the presence of a zinc oxide catalyst at a temperature of about 230˜240° C., at a pressure of 200 torr. Then the gas stream is cooled at a temperature of about −78˜10° C. to separate unreacted lactic acid from a mixture. Subsequently, the mixture is mixed with water to separate D-type lactide from the mixture.

The above and other features of the invention are discussed infra.

### BRIEF DESCRIPTION OF THE DRAWINGS

The above and other features of the present invention will now be described in detail with reference to certain exemplary embodiments thereof illustrated the accompanying drawings which are given hereinbelow by way of illustration only, and thus are not limitative of the present invention, and wherein:

FIG. 1 illustrates an exemplary process of producing lactide from lactic acid and separating impurities.

It should be understood that the appended drawings are not necessarily to scale, presenting a somewhat simplified representation of various preferred features illustrative of the basic principles of the invention. The specific design features of the present invention as disclosed herein, including, for example, specific dimensions, orientations, locations, and shapes will be determined in part by the particular intended application and use environment.

In the figures, reference numbers refer to the same or equivalent parts of the present invention throughout the several figures of the drawing.

### DETAILED DESCRIPTION

Hereinafter reference will now be made in detail to various embodiments of the present invention, examples of which are illustrated in the accompanying drawings and described below. While the invention will be described in conjunction with exemplary embodiments, it will be understood that present description is not intended to limit the invention to those exemplary embodiments. On the contrary, the invention is intended to cover not only the exemplary embodiments, but also various alternatives, modifications, equivalents and other embodiments, which may be included within the spirit and scope of the invention as defined by the appended claims.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about. In a exemplary embodiment of the present invention a method of producing D-type lactide begins by converting liquid D-type lactic acid into D-type polylactic acid having a weight average molecular weight of about 600˜1200 g/mol at a temperature of about 130˜150° C. and a pressure of 10˜200 torr. Next, the D-type polylactic acid having a weight average molecular weight of about 600˜1200 g/mol is converted into a gas stream by heating the D-type polylactic acid in the presence of a zinc oxide catalyst at a temperature of about 230˜240° C., at a pressure of 200 torr. Then the gas stream is cooled at a temperature of about −78˜10° C. to separate unreacted lactic acid from a mixture. Subsequently, the mixture is mixed with water to separate D-type lactide from the mixture

For understanding of the present invention, there will be given definitions according to the structure of lactic acid:

L1A: lactic acid or lactic acid monomer or 2-hydroxy propionic acid

LD: lactide or 3,6-dimethyl-1,4-dioxane-2,5-dione (circular structure)

L2A: lactoyl lactic acid or linear lactic acid dimer

L3A: lactoyl lactoyl lactic acid or linear lactic acid trimer

LnA: n-oligomer of linear lactic acid

The DP or degree of polymerization of lactic acid is “n”, that is, the number average of lactic acid units that are covalently attached in the lactic acid polymer. Lactic acid has one asymmetric carbon atom, and thus can be found in two enantiomeric forms. Lactide, on the other hand, has two asymmetric carbon atoms so that it can be found in three stereoisomeric forms: L-lactide in which both asymmetric carbon atoms possess the L (or S) configuration; D-lactide in which both asymmetric carbon atoms possess the D (or R) configuration; and meso-lactide in which one asymmetric atom has the L configuration and the other has the D configuration.

L-lactide and D-lactide are enantiomers. In the production of lactide from lactic acid, it would be advantageous if the absolute configuration of the lactic acid feed was maintained in its conversion to lactide.

A step (a) of converting liquid D-type lactic acid into D-type polylactic acid having a weight average molecular weight of about 600˜1200 g/mol at a temperature of about 130˜150° C. and a pressure of about 10˜200 torr is a step of performing condensation polymerization of liquid D-type lactic acid under reduced pressure to synthesize low molecular weight D-type polylactic acid. It is preferable that the produced low molecular weight polylactic acid mainly includes L2A and L3A.

A step (b) of converting the D-type polylactic acid having a weight average molecular weight of about 600˜1200 g/mol into a gas stream by heating it in the presence of a zinc oxide catalyst at a temperature of about 230˜240° C. and at a pressure of about 200 torr is a step of converting a part of polylactic acid into D-type lactide while converting D-type polylactic acid into a gas stream by heating the D-type lacpolylactic acid in the presence of a zinc oxide catalyst. Upon heating, it is preferable to maintain the temperature at about 230˜240° C. at the pressure of about 200 torr. For example, if the temperature is less than about 230° C., the extent of depolymerization is dramatically reduced to rapidly decrease the yield. If the temperature is more than about 240° C., initial depolymerization actively occurs, but carbonization as well as changes in the color and appearance of the low molecular weight polylactic acid may problematically occur.

The zinc oxide catalyst may exist in an amount of about 0.01˜1.5% by weight, based on the D-type polylactic acid. It is preferable that a carrier gas is added and the retention time is controlled from about 5˜10 sec in step (b), and the carrier gas may include nitrogen gas.

In step (c) of cooling the gas stream at a temperature of about −78˜10° C. to separate unreacted polylactic acid and a mixture, unreacted polylactic acid and the mixture containing D-type lactide may be separated through a cold cyclone for separation.

Step (d) of mixing the mixture with water to separate lactide is a step of mixing the mixture containing D-type lactide separated in step (c) with water in a separation vessel to remove a liquid containing water and impurities formed at the top and to recover only lactide crystals from the bottom. The mixing may be performed at a temperature of about 5˜30° C. The volume ratio of the mixture and water may be about 1:0.5˜5. The unreacted lactic acid separated in step (c) is recycled to step (a), and thus the process may be a continuous cyclic process.

The produced D-type lactide may be produced into D-type polylactic acid having a weight average molecular weight of about 50,000˜20,000 g/mol using one or more catalysts selected from the group consisting of tin, tin halides, tin carboxylates, and tin alkoxides and alcohols having about 1˜12 carbon atoms at a temperature of about 150˜200° C.

### EXAMPLES

The following examples illustrate the invention and are not intended to limit the same.

### Examples 1, 2

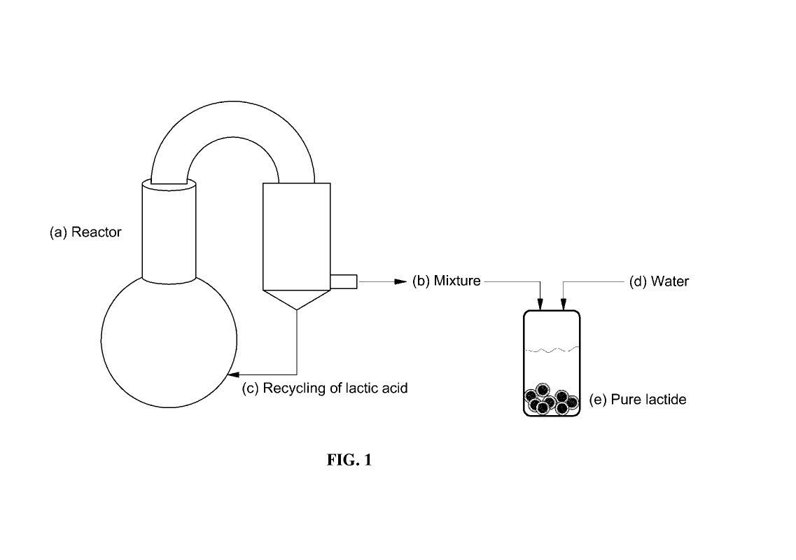

The laboratory scale reactor used in the Examples is illustrated in FIG. 1. The to reactor is a reactor having a width of approximately 5 cm and a height of approximately 5 cm. A transport line is connected to the top of the reactor.

The bottom portion of the reactor was charged with liquid D-type lactic acid, and a zinc oxide catalyst was injected into the liquid lactic acid. The reactor was equipped with a Tee-valve at the bottom, thereby supplying the liquid lactic acid to the reactor. A nitrogen carrier gas was also supplied to the reactor through a line connected to the Tee. This entire assembly was placed in a heating mantle which was heated to maintain the desired reactor temperature. Lactide and other by-products produced by catalyst reaction in the reactor were vaporized.

The catalyst added to the liquid lactic acid was zinc oxide (ZnO), and it was added in an amount of 0.5% by weight (Example 1) and 1.0% by weight (Example 2), based on the liquid lactic acid. Subsequently, unreacted lactic acid and a mixture containing lactide were separated through the cold cyclone.

The mixture containing the obtained lactide and impurities were transported to a separation vessel, and then mixed with water. Water was added in an equal volume to the total volume of lactide and impurities, and the mixing was performed at a temperature of about 15° C. for 1 hr. The upper liquid was removed, and on the D-type lactide crystals at the bottom were recovered. More detailed reaction conditions are described in the following Table 1.

### Comparative Example

This process was performed in the reactor and flow tube of Example 1 in the same manner, except that the catalyst was not introduced to the reactor. In Comparative Examples 4˜6, a separation process of the mixture was not performed. More detailed reaction conditions are described in the following Table 1.

TABLE 1

| | Example | | Comparative Example | | | | | |
|---|---|---|---|---|---|---|---|---|
| Section | 1 | 2 | 1 | 2 | 3 | 4 | 5 | 6 |
| Zinc oxide | 0.5 | 1.0 | 0.5 | 1.0 | 0.5 | x | x | x |
| catalyst (% by | | | | | | | | |
| weight) | | | | | | | | |
| Depolymeriza- | 230 | 240 | 210 | 200 | 260 | 250 | 260 | 270 |
| tion | | | | | | | | |
| temperature | | | | | | | | |
| (° C.) | | | | | | | | |
| Pressure (torr) | 200 | 200 | 200 | 200 | 200 | 200 | 200 | 200 |
| Mixture | ∘ | ∘ | ∘ | ∘ | x | x | x | x |
| Separation | | | | | | | | |
| process using | | | | | | | | |
| water | | | | | | | | |
| Mixture | x | x | x | x | ∘ | x | x | x |
| Separation | | | | | | | | |
| process using | | | | | | | | |
| ethanol | | | | | | | | |
| Final | 65 | 72 | 15 | 25 | 35 | 35 | 34 | 36 |
| production | | | | | | | | |
| yield of lactide | | | | | | | | |
| (%) | | | | | | | | |

As shown in Table 1, when the production process of the present invention was applied, lactide particles can be produced from lactic acid at a remarkably high yield.

### Example 3

3000 g of the D-type lactide produced in Example 1 was introduced into a reactor equipped with a stirrer, and heated to 300° C. under nitrogen atmosphere. 0.9 g of stannous octoate and 1.8 g of 1-hexanol were introduced thereto. Subsequently, the reaction was performed for 2 hrs at a temperature of 180° C. to recover polymers from the reactor. A to pulverization process was performed to give D-type polylactic acid having a weight average molecular weight of approximately 150,000 g/mol.

### Effect of the Invention

The method of the present invention is advantageous in that D-type lactide can be obtained at a high yield by a simple method, compared to the conventional production methods. Consequently, production cost of D-type polylactic acid that is finally obtained from D-type lactide can be reduced.

The method of the present invention is also advantageous in that the asymmetric carbon atoms in the product lactide predominate in the same absolute configuration as the liquid phase lactic acid from which it was made, and unreacted aqueous lactic acid can be recycled, and by-products are hardly formed.

The lactide produced by the method of the present invention can be used as a raw material for the production of D-type polylactic acid. The produced D-type polylactic acid forms a stereocomplex together with L-type polylactic acid, and thus has high heat resistance and impact resistance, thereby being used as an alternative biomass material to the conventional petroleum-based polypropylene materials. In particular, it can be applied to automotive interior/exterior parts. Therefore, considering the recent trend of high oil prices, dependence on petroleum-based products can be reduced, and production costs for interior and exterior materials can be also greatly reduced.

While this invention has been described in connection with what is presently considered to be practical exemplary embodiments and example, it is to be understood that the invention is not limited to the disclosed embodiments, but, on the contrary, is intended to cover various modifications and equivalent arrangements included within the spirit and scope of the appended claims.

What is claimed is:

1. A method of producing D-type lactide, comprising the steps of:
(a) converting liquid D-type lactic acid into D-type polylactic acid having a weight average molecular weight of about 600˜1200 g/mol at a temperature of about 130˜150° C. and a pressure of about 10˜200 torr;
(b) converting the D-type polylactic acid having a weight average molecular weight of about 600˜1200 g/mol into gas stream by heating D-type polylactic acid in the presence of a zinc oxide catalyst at a temperature of about 230˜240° C. and at a pressure of 200 torr;
(c) cooling the gas stream at a temperature of about −78˜10° C. to separate unreacted lactic acid from a mixture; and
(d) mixing the mixture with water to separate D-type lactide.

2. The method of claim 1, wherein the zinc oxide catalyst exists in an amount of about 0.01˜1.5% by weight, based on D-type polylactic acid.

3. The method of claim 1, wherein a carrier gas is added and the retention time is controlled from about 5˜10 sec in step (b).

4. The method of claim 3, wherein the carrier gas includes a nitrogen gas.

5. The method of claim 1, wherein the mixture and water are mixed at a volume ratio of about 1:0.5˜5 and at a temperature of about 5˜30° C.

6. The method of claim 1, wherein the unreacted lactic acid separated in step (c) is recycled to step (a) to create a continuous cycle.

7. A method of producing D-type polylactic acid having a weight average molecular weight of about 50,000˜20,000 g/mol from the D-type lactide the method comprising:
converting liquid D-type lactic acid into D-type polylactic acid having a weight average molecular weight of about 600˜1200 g/mol at a temperature of about 130˜150° C. and a pressure of about 10˜200 torr;
converting the D-type polylactic acid having a weight average molecular weight of about 600˜1200 g/mol into gas stream by heating D-type polylactic acid in the presence of a zinc oxide catalyst at a temperature of about 230˜240° C. and at a pressure of 200 torr;
cooling the gas stream at a temperature of about −78˜10° C. to separate unreacted lactic acid from a mixture;
mixing the mixture with water to separate D-type lactide;
using the separated D-type lactide to produce the D-type polylactic acid by using one or more catalysts selected from the group consisting of tin, tin halides, tin carboxylates, and tin alkoxides and alcohols having about 1˜12 carbon atoms at a temperature of about 150˜200° C.
